(19) United States
(12) Patent Application Publication
(10) Pub. No.: US 2022/0192965 A1
(43) Pub. Date: Jun. 23, 2022

(54) COSMETIC COMPOSITION FOR HAIR

(71) Applicant: SPCM SA, Andrezieux Boutheon (FR)

(72) Inventors: Frédéric BLONDEL, Andrezieux Boutheon (FR); Thomas BOURSIER, Andrezieux Boutheon (FR); Charlène BOTTE, Andrezieux Boutheon (FR)

(73) Assignee: SPCM SA, Andrezieux Boutheon (FR)

(21) Appl. No.: 17/441,884

(22) PCT Filed: Mar. 16, 2020
(86) PCT No.: PCT/FR2020/050562

§ 371 Date: Sep. 22, 2021

(30) Foreign Application Priority Data
Apr. 3, 2019 (FR) 1903581

(51) Int. Cl. A61K 8/81; A61Q 5/06; A61K 8/04

(52) U.S. Cl. CPC A61K 8/8147; A61Q 5/06; A61K 2800/95; A61K 2800/48; A61K 8/042

(57) ABSTRACT

The invention relates to an aqueous cosmetic composition for care and maintenance of the hair comprising at least one setting resin, said resin comprising at least 20 mol % of a monomer unit of methacrylic acid and/or one of its salts; and optionally at least one monomer unit of acrylic acid and/or maleic anhydride, and/or one of their salts, the resin having a weight-average molecular weight greater than or equal to 20,000 g/mol. The invention also relates to the use of the setting resin for improving the resistance to humidity of these cosmetic compositions.

### FIELD OF THE INVENTION

This invention relates to the technical field of hair care and styling by means of cosmetic compositions.

The subject-matter of the invention is an aqueous cosmetic composition comprising a setting resin of a specific chemical nature and the use of this resin to improve the resistance to humidity of said composition.

### PRIOR ART

The setting agents used in hair gels are usually film-forming polymers that bind the hair together, thus making it easier to style. The setting agents usually used are natural gums, such as guar gum or anionic or neutral polymers.

To be effective, they must meet a certain number of criteria, mainly guided by consumer perception. For example, the film must be transparent, more or less flexible depending on the desired effect, but must have a certain strength, and a certain robustness. Likewise, the film must not flake over time or under stress (combing, friction) and must not become sticky, when weather conditions become humid.

Furthermore, these polymers, otherwise known as resins, must make it possible to formulate clear compositions with rheological characteristics in line with the packaging (aerosol, tube, jar) and consumer expectations (gel-like viscosity, pseudoplastic, non-sticky, quick-drying).

Moreover, in general, cosmetic compositions based on these polymers contain thickeners, which provide the rheological properties mentioned above.

Document WO 99/13836 describes solvent-based hair cosmetic compositions containing a reduced level of volatile organic compound (VOC) to meet environmental requirements. These compositions are based on a specific solvent combination comprising ethanol associated with either methyl acetate or butyl acetate. They also comprise a setting resin which, according to the examples disclosed, may be selected from a series of polymers, and in particular from an octylacrylamide/acrylates/butylaminoethyl methacrylate copolymer, a vinyl acetate/crotonates/vinyl neodecanoate copolymer, a diglycol/cyclohexane dimethanol/isophthalates/sodium sulfoisophthalate copolymer. However, the document does not indicate the molar percentages of monomers or the molecular weight of the polymers. Some of the alcohol may also be replaced by water, but in this case, the stability of the composition against freeze/thaw cycles is degraded.

Document EP 0 761 199 describes cosmetic compositions for the hair comprising a setting agent and a particular acrylic copolymer. The setting agent is selected from anionic, cationic, or amphoteric polymers, and copolymers of acrylic or methacrylic acid are mentioned among the list of anionic polymers without specifying either their chemical composition or their molecular weight. Acrylic copolymer is a tetrapolymer composed of alkyl acrylate, alkyl methacrylate, acrylic acid, and methacrylic acid. The total quantity of the two acrylic acid and methacrylic acid monomers does not exceed 15% by weight. This tetrapolymer has a molecular weight of between 10,000 and 50,000 gm/mol. These compositions have an acceptable resistance to humidity, but nevertheless, this may be improved.

The scientific publication “High Molecular Weight Poly (methacrylic acid) with Narrow Polydispersity by RAFT Polymerization”—Macromolecules, 2009, 42, pages 1494-1499, describes the preparation of a homopolymer of methacrylic acid, i.e., containing 100 mol % monomer units of methacrylic acid.

Document EP 0 589 241 describes a polymer used in the field of printed circuits, comprising at least 20 mol % of monomer units of methacrylic acid, monomers of methyl methacrylate, and monomers of ethyl acrylate. This polymer is used in solution in methyl ethyl ketone. The polymer is not water-soluble and, therefore, not suitable for an aqueous composition.

Document EP 2 248 510 describes thickening polymers and their use in aqueous compositions. Said aqueous composition comprises two thickening polymers, including a first thickening polymer comprising from 10% to 50% by weight of methacrylic acid and a second thickening polymer not comprising methacrylic acid.

Document EP 1 402 877 describes aqueous compositions comprising a surfactant, clay, and a copolymer comprising monomers of acrylic acid, of alkyl methacrylate, of modified methacrylate, and optionally of methacrylic acid.

Document U.S. Pat. No. 4,196,190 describes a setting resin for the hair. This resin comprises a copolymer comprising monomers of methacrylic acid, methyl methacrylate, hydroxyethyl methacrylate, and alkyl acrylate. The percentage of monomers of methacrylic acid in the copolymer is preferably low, i.e., less than or equal to 18% by weight or 21.5 mol %, so as to not degrade curl retention. Document U.S. Pat. No. 4,543,249 describes a copolymer and its use in a composition for the hair. This copolymer comprises from 70% to 90% by weight of methyl methacrylate and from 10% to 30% by weight of methacrylic acid and/or one of its salts.

Nevertheless, there is a need to develop new polymers with the objective of improving the properties of moisture resistance in setting resins for their use in aqueous cosmetic compositions for hair care while making it possible to advantageously formulate transparent compositions, having excellent rheological properties, and without exhibiting the drawbacks mentioned above.

### DISCLOSURE OF THE INVENTION

The Applicant has now discovered, surprisingly and unexpectedly, that a specific selection of a polymer, by the nature and the amounts of monomers constituting it, as well as a judicious choice of molecular weight, made it possible to achieve the objectives described above.

This invention, therefore, provides an aqueous cosmetic composition for hair care that offers excellent holding properties in a humid environment for several hours without becoming sticky and that is easy to remove during washing.

More specifically, this invention relates to an aqueous cosmetic composition for hair care comprising at least one setting resin, said resin comprising:
at least 30 mol % of monomer units of methacrylic acid and/or one of its salts;
at least 10 mol % of monomer units of acrylic acid and/or maleic acid and/or itaconic acid, and/or a salt thereof;
   the resin being water-soluble and having a weight-average molecular weight greater than or equal to 20,000 gm/mol (≥20,000 gm/mol).

The sum of the molar percentages of the monomer units of the resin, including, in particular, the monomer units of methacrylic acid and the monomer units of acrylic acid and/or monomer units of maleic acid and/or itaconic acid, is equal to 100%. The person skilled in the art will know how to adjust the respective percentages to 100%, with at least 30 mol % of methacrylic acid and/or one of its salts and at least 10 mol % of acrylic acid and/or maleic acid and/or itaconic acid, and/or one of their salts.

According to one embodiment of the invention, said resin consists of, or essentially consists of:
at least 30 mol % of monomer units of methacrylic acid and/or one of its salts;
of at least 10 mol % of monomer units of acrylic acid and/or maleic acid and/or itaconic, and/or one of their salts;
the resin being water-soluble and having a weight-average molecular weight greater than or equal to 20,000 gm/mol (≥20,000 gm/mol).

The term “consisting of” means that the resin comprises only monomer units of methacrylic acid, and/or a salt thereof, at least 30% molar, and monomer units of acrylic acid and/or acid maleic and/or itaconic acid, and/or one of their salts, at least 10 mol %. The sum of these molar percentages is then equal to 100%.

This invention also relates to the use as a setting agent of this resin in an aqueous cosmetic composition for hair care, in particular for improving the resistance to humidity of this cosmetic composition.

Setting Resin

In the context of this invention, the term setting resin is understood to mean a polymer making it possible to give a cosmetic composition a temporary holding effect, setting the hairstyle but also retaining curl, even in a humid environment. Hereinafter, the setting resin will be used to designate the “dry” polymer and not the solution containing the polymer. Most often, a solution containing the polymer is used; said solution generally containing 15 to 25% of polymer by weight relative to the weight of the solution. This is called a solution containing 15 to 25% of active material by weight relative to the weight of the solution. Thus, the amounts of setting resin will be given as a percentage by weight of active material, unless this is contraindicated.

According to a preferred embodiment of the invention, the setting resin comprises at least 30 mol % of monomer units of methacrylic acid and/or one of its salts, preferably at least 40 mol %, more preferably at least 50 mol %, again more preferably at least 60 mol % and even more preferably at least 70 mol %.

According to a preferred embodiment of the invention, the setting resin comprises less than 99 mol % of monomer units of methacrylic acid and/or one of its salts, preferably less than 95 mol %, more preferably less than 90 mol %, even more preferably less than 80 mol %.

The salts of methacrylic acid include, in particular, the salts of an alkaline earth metal (preferably calcium or magnesium) or of an alkali metal (preferably sodium or lithium) or of ammonium (in particular quaternary ammonium).

According to a preferred embodiment of the invention, the setting resin comprises at least 1 mol % of monomer units of acrylic acid and/or maleic acid and/or itaconic acid and/or one of their salts preferably at least 10 mol %, more preferably at least 20 mol %.

According to a preferred embodiment of the invention, the setting resin comprises less than 80 mol % of monomer units of acrylic acid and/or maleic acid and/or itaconic acid and/or one of their salts, preferably less than 70 mol %, more preferably less than 60 mol %, even more preferably less than 50 mol %, still more preferably less than 40 mol % and even more preferably less than 30 mol %.

The term “molar percentage” is understood to mean molar percentages relative to all the moles of monomer of the resin or of the polymer. On the other hand, when the resin (or the polymer) is cross-linked or branched, the amount of branching agent or of cross-linking agent is not taken into account to determine all the moles of the resin's monomer (or of the polymer).

According to a preferred embodiment of the invention, the setting resin comprises monomer units of methacrylic acid and/or one of its salts, monomer units of acrylic acid and/or maleic acid and/or itaconic acid and/or one of their salts, and optionally nonionic monomers and/or cationic monomers. Even more preferably, the setting resin comprises monomer units of methacrylic acid and/or one of its salts, monomer units of acrylic acid and/or one of its salts, and optionally nonionic monomers and/or cationic monomers.

The setting resin is preferably a copolymer of methacrylic acid and of acrylic acid and/or of their salts. In other words, the resin is a copolymer comprising only monomer units of methacrylic acid, and of acrylic acid, and/or of their salts.

The following monomers: methacrylic acid, acrylic acid, maleic acid, and itaconic acid can be in acid form (—C(═O)—OH) or in salt form (—C(═O)—O⁻; Cation) of alkaline earth metal, (preferably calcium or magnesium), of alkali metal (preferably sodium or lithium) or of ammonium (in particular quaternary ammonium). The preferred salts are the sodium salts.

The weight-average molecular weight of the resin is preferably greater than or equal to 60,000 gm/mol, more preferably greater than or equal to 100,000 gm/mol. It is preferably less than or equal to 2,000,000 gm/mol (≤2 million gm/mol), more preferably less than or equal to 1,000,000 gm/mol, even more preferably less than or equal to 500,000 gm/mol, even more preferably less than or equal to 300,000 gm/mol.

The average molecular weight is determined, in a conventional manner, by size exclusion chromatography. For example, it is measured on an Agilent 1260 Infinity system equipped with a Dawn HELOS, OPtilab T-Rex multi-angle light scattering detector, and 3 columns in series: Shodex SB 807-G, Shodex SB 807-HQ, and Shodex 805-HQ.

The setting resin is preferably a water-soluble polymer. The term “water-soluble polymer” is understood to be a polymer that gives an aqueous solution when it is dissolved with stirring at 25° C. at a concentration of 20 gm L⁻¹ in water.

The setting resin may also comprise at least nonionic and/or cationic monomer units. In this case, it is preferable to limit the amount of these monomers to less than 20 mol %, preferably to less than 10 mol %.

The nonionic monomers are preferably chosen from the group comprising acrylamide, methacrylamide, N-vinylformamide, and N-vinylpyrrolidone. Acrylamide is preferred.

Advantageously, the setting resin is devoid of hydroxylated monomers, for example of hydroxypropyl acrylate and/or alkyl (meth) acrylate type.

The cationic monomers are preferably chosen from the group comprising quaternized or salified dimethylaminoethyl acrylate (ADAME); quaternized or salified dimethylaminoethyl methacrylate (MADAME); dimethyldiallylammonium chloride (DADMAC); acrylamido propyltrimethyl ammonium chloride (APTAC); and methacrylamido propyltrimethyl ammonium chloride (MAPTAC).

Those skilled in the art will know how to prepare the quaternized or salified ADAME and MADAME monomers, for example, by means of acid (in particular hydrochloric acid) or of an alkyl halide of the RX type, R being an alkyl group and X being a halogen (especially methyl chloride). It is also possible to use a branching agent or a cross-linking agent. Such an agent is, for example, chosen from methylene-bis-acrylamide (MBA), ethylene glycol diacrylate, tetraallyl ammonium polyethylene glycol chloride, diacrylamide, cyanomethyl acrylate, epoxides, and mixtures thereof. It is also possible to use a free radical chain transfer agent, also known as a chain limiter. The use of a chain transfer agent is particularly advantageous for controlling the molecular weight of the polymer obtained. Examples of transfer agents include methanol, isopropanol, sodium hypophosphite, 2-mercaptoethanol, sodium methallylsulphonate, and their mixtures.

Those skilled in the art will know how to adjust the amounts of branching agent and optionally of a transfer agent so as to preferably obtain a water-soluble resin.

In general, the resin or polymer does not require the development of a particular polymerization process. Indeed, it may be obtained according to all the polymerization techniques well known to those skilled in the art. In particular, it may be solution polymerization; gel polymerization; precipitation polymerization; emulsion polymerization (aqueous or reverse); suspension polymerization; reactive extrusion polymerization; water-in-water polymerization; or micellar polymerization.

In the context of the invention, the preferred polymerization technique is solution polymerization. It indeed makes it possible to obtain polymers having a molecular weight in line with the desired effects and benefits.

Polymerization is generally free radical polymerization. In particular, free radical polymerization is meant to be understood as free radical polymerization by means of UV, azo, redox, or thermal initiators, as well as controlled radical polymerization (CRP) techniques or matrix polymerization techniques.

Aqueous Cosmetic Composition for Hair Care and Styling

In the context of this invention, an aqueous composition denotes a composition containing water in a proportion such that the main phase is aqueous.

The presence of the setting resin does not alter the clarity of the aqueous cosmetic composition according to the invention. Indeed, an aqueous composition consisting of water and setting resin is advantageously transparent.

The term “transparent composition” is meant to be understood as a composition whose measured transmittance value at 420 nm is greater than 85%. The transmittance measurement is carried out at 420 nm, for example, in a cell (advantageously a 1.5 mL polystyrene cell), with an optical path of 10 mm, at 25° C., for a solution comprising 2.4 gm/L of resin in water. This measurement is carried out by means of a spectrophotometer, for example, a spectrophotometer of the Hach Lange DR 600 type.

The composition according to the invention may be in the form of a gel, a solution, a spray, or hair spray without this being a limiting factor. In a preferred embodiment, the composition is in the form of a gel. It is advantageously transparent according to the definition above.

The aqueous cosmetic composition according to the invention preferably comprises between 0.05 and 10% by weight of the setting resin, preferably between 0.1 and 5% by weight, even more preferably between 0.15 and 1% by weight.

In a preferred embodiment, the composition comprises:
water;
from 0.05 to 10% by weight of setting resin according to the invention;
a thickening agent.

As already indicated, this aqueous cosmetic composition is advantageously transparent.

The composition according to the invention comprises water. It may also comprise a mixture of water and cosmetically acceptable solvents such as monoalcohols, polyalcohols, glycol ethers. More in particular, mention may be made of lower alcohols such as ethanol, isopropanol, polyalcohols such as diethylene glycol, glycol ethers.

The water concentration is generally between 50 and 99% by weight relative to the total weight of the composition. It is preferably at least 60% by weight, preferably at least 80%, and even more preferably at least 90%.

According to the invention, any type of thickening agent may be used, including anionic, cationic, and amphoteric polymers, with a preference for cross-linked acrylic polyacids. Carbomers are particularly preferred. Their amount is generally between 0.01 and 10% by weight, preferably between 0.05 and 5% by weight.

The pH of the compositions according to the invention is generally between 2 and 9, and in particular between 3 and 8. It may be adjusted to the value selected by using basifying or acidifying agents usually used in cosmetics for this type of application.

When the composition according to the invention is pressurized in aerosol form in order to obtain a hair spray, it comprises at least one propellant, which may be chosen from volatile hydrocarbons such as n-butane, propane, isobutane, pentane, chlorinated, and/or fluorinated hydrocarbons and mixtures thereof. Carbon dioxide, nitrous oxide, dimethyl ether, nitrogen, compressed air, and their mixtures may also be used as propellants.

The propellant concentration is generally between 10 and 50% by weight relative to the total weight of the pressurized composition.

The compositions according to the invention, pressurized or not, may also contain surfactants, preservatives, sequestering agents, softeners, colorants, viscosity modifiers, foam modifiers, anti-foam agents, agents pearlescent agents, moisturizers, anti-dandruff agents, anti-seborrheic agents, sunscreens, proteins, vitamins, plasticizers, hydroxy acids, and perfumes.

The compositions according to the invention may also contain other conditioning agents. These may then be selected from natural or synthetic oils and waxes, fatty alcohols, esters of polyhydric alcohols, glycerides, silicone gums, and resins or mixtures of these various compounds.

Of course, a person skilled in the art will take care to choose the optional compound(s) to be added to the composition according to the invention in such a way that the advantageous properties inherently attached to the composition in accordance with the invention are not, or are substantially not, altered by the planned addition.

For example, the hair care compositions used according to the invention are gels or solutions designed to be rinsed out or left in. For example, they are waving lotions, blow-drying lotions, setting compositions (lacquers), and styling compositions. The lotions may be packaged in various forms, such as spray bottles, pump bottles, or aerosol containers to ensure application of the composition in spray form. For example, compositions in gel form are packaged in jars, which are preferably transparent.

This invention will be described in more detail with reference to the following examples. They illustrate the invention simply and are non-limiting.

### FIGURES

FIG. 1 illustrates the change in the percentage of curl retention over time of the compositions containing a setting resin.

### EXAMPLES OF EMBODIMENTS OF THE INVENTION

### 1/Synthesis of Setting Resins

203.0 gm of water and 50 mg of sodium hypophosphite are introduced into a jacketed reactor equipped with a mechanical stirrer with a half-moon paddle, a refrigerant, and a temperature probe. The medium is heated to 83° C. When the temperature is reached, a solution containing acrylic acid (AA) and methacrylic acid (AMA) or hydroxypropyl acrylate (HPA) co-monomer is added over a period of 120 minutes. The quantities of AA and AMA or HPA are shown in Table 1.

In parallel, a 10.0% by weight sodium persulfate solution is added over a period of 135 minutes. When pouring of the sodium persulfate solution has ended, the medium is cooled to 60° C., and 156.8 gm of water is added to the medium. The pH is adjusted between 2.2 and 3.0 then the medium is stirred for an additional hour. The product is then filtered at 300 μm.

Polymeric solutions corresponding to Examples 2 to 6 are obtained, the compositions and the average molecular weights by weight (Mw) of which are detailed in Table 1. The resin of the composition of Example 1, for its part, contains only acrylic acid monomers.

TABLE 1
Compositions and Molecular Weight of Setting Resins

| | Polymer composition (mol %) | | | Mw |
|---|---|---|---|---|
| | AA | AMA | HPA | (gm/mol) |
| Example 1 (Comparative) | 100 | | | 191,000 |
| Example 2 (Comparative) | 91.5 | 8.5 | | 172,000 |
| Example 3 (Invention) | 55 | 45 | | 152,000 |
| Example 4 (Invention) | 29 | 71 | | 179,000 |
| Example 5 (Comparative) | 55 | 45 | | 15,900 |
| Example 6 (Comparative) | 55 | | 45 | 326,000 |

### 2/Manufacture of Transparent Aqueous Cosmetic Compositions for Hair Care

The resins corresponding to Examples 1 to 6 are incorporated into the gel formulation described in Table 2. We thus obtain compositions C′1 to C′6 containing respectively setting resins 1 to 6.

TABLE 2
Hair Gel Composition

| | % by Weight |
|---|---|
| Water | Qs 100 |
| Carbomer (Flogel FG 1000) | 2.00 |
| Polymer of Examples 1 to 7 | 0.24 |
| Glycerine | 1 |
| EDTA | 0.03 |
| TEA | Qs pH 6.5 |
| Perfume | 0.2 |

Qs 100=sufficient quantity to reach 100%

EDTA=ethylenediaminetetraacetic acid

TEA=Triethanolamine

Qs pH 6.5=sufficient quantity to reach a pH of 6.5

The clarity of the gels is determined by measuring the transmittance of light on a Hach Lange DR 600 spectrophotometer. These measurements were carried out at 420 nm in 1.5 mL PS cuvettes, at 25° C., for a solution comprising 2.4 gm/L of resin in water.

The viscosity of the gels is determined at 23° C. with a Brookfield RVT viscometer at a speed of 20 revolutions/min. Those skilled in the art know how to choose the modulus as a function of the viscosity of the gels. In this case, and taking into account the composition of the gel and the desired texture, module 7 is used.

The results are reported in Table 3.

TABLE 3
Viscosity and Clarity of the Styling Gels

| Gel Composition | Viscosity CPS (20 tpm) | Clarity % T (420 nm) |
|---|---|---|
| C′1 (Comparative) | 54.000 | 89 |
| C′2 (Comparative) | 56.000 | 91 |
| C′3 (Invention) | 58.000 | 92 |
| C′4 (Invention) | 64.000 | 91 |
| C′5 (Comparative) | 70.000 | 97 |
| C′6 (Comparative) | 61.000 | 88 |

The gels of compositions C′1 to C′6 obtained are all transparent because they have a clarity greater than 85% T. Their viscosity and their rheological property are very good.

### 3/Resistance to Humidity of the Setting Resins

The resins' resistance to humidity was evaluated by a “curl retention” test.

The protocol is as follows: 3.1 gm of resin is added to 87.5 gm of hydroalcoholic water/ethanol solution (37/63). We thus obtain the compositions C′1 to C′6 containing the resins of Examples 1 to 6, respectively.

A strand of European-type hair is immersed in the hydroalcoholic resin solution, and the excess solution is removed by passing the strand of hair between two fingers over its entire length. The hair strand is then combed once and rolled up on a curler.

The strands are then placed on the curler for 24 hours at 23° C. and 50% relative humidity. The strands are unrolled and placed in a humidity-controlled oven at 90% at 25° C. The length of the strand at to is used as a reference (Li) for the calculation of the percentage of curl hold (% TB), also known as the percentage of curl retention (% CR). The length of the strands is measured at different time intervals (Lt).

The percentage of curl retention is calculated using the following ratio:

% TB=[(L−Lt)/(L−Li)]*100

with L=Length of the unrolled strand=19 cm.

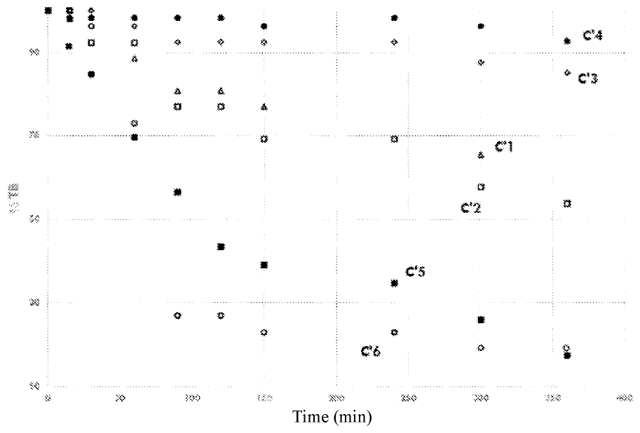

We observe, in FIG. 1, that the compositions C′3 and C′4, respectively containing the resins of Examples 3 (45% mol AMA) and 4, (71% mol AMA), offer excellent resistance to humidity, although greater than those obtained with compositions C′1 and C′2, containing the resins of Examples 1 (0 mol % AMA) and 2 (8.5 mol % AMA). Composition C′2 exhibits poorer curl retention than composition C′1. This demonstrates that the incorporation of AMA in a resin is not the only parameter for increasing curl strength but that a minimum molar percentage of at least 30 AMA in the AA/AMA copolymer is necessary to obtain better curl retention.

We also observe from the results of compositions C′3 and C′4 in FIG. 1 that the increase in the amount of AMA in the copolymer makes it possible to obtain increased resistance to humidity. Furthermore, compositions C′5 and C′6 offer very poor resistance to humidity. The observation of curve C′6 compared to curve C′3 allows us to say that the substitution of AMA by HPA strongly degrades the resistance to humidity.

Observation of curve C′5 compared to curve C′3 makes it possible to demonstrate the importance that the molecular weight of the resin has on the resistance to humidity of the composition containing it. In fact, composition C′5 containing an AA/AMA 45/55 copolymer with a weight-average molecular weight of less than 20,000 gm/mol does not make it possible to increase curl retention. Finally, the strands prepared with compositions C′3 and C′4 according to the invention do not exhibit any stickiness and are easy to remove during washing, thus satisfying the desired criteria.

### 4/Examples 7 and 8

A methacrylic acid homopolymer (Example 7), i.e., containing 100 mol % of monomer units of methacrylic acid, is prepared according to the method described in the scientific publication “High Molecular Weight Poly(methacrylic acid) with Narrow Polydispersity by RAFT Polymerization”—Macromolecules, 2009, 42, pages 1494-1499. The polymer obtained corresponds to example 7. Another polymer is prepared according to the same method, except that 12 mol % of the methacrylic acid is replaced by 12 mol % of acrylic acid. The polymer obtained corresponds to Example 8. The polymer of Example 7 has such high viscosity that it cannot be used by formulators of cosmetic compositions due to the fact that it is too difficult to handle. Nor does a dilution test to reduce the viscosity lead to a product that is satisfactory for the user because of stability problems. Its properties are therefore not evaluated.

The polymer of Example 8 is able to be handled. A hair gel composition C′8 is made with the polymer of Example 8 according to the formulation of Table 2. The viscosity of the gel is 52,000 CPS, and the clarity is 91%. The same resistance to humidity test as in part 3/is carried out, and the curl hold at 300 minutes is 93%, which is very satisfactory and demonstrates the technical effect of the polymers of the invention.

### 5/Examples 9 to 14

The polymers of lines 2, 5, and 8 of Table 1 of document EP1402877 are produced. They are noted as examples 9, 11, and 13, respectively. A polymer 10 is made as polymer 9, in which 10 mol % ethyl acrylate is replaced by 10 mol % acrylic acid. A polymer 12 is made as polymer 11, in which 25 mol % ethyl acrylate is replaced by 25 mol % methacrylic acid. A polymer 14 is made, such as polymer 13, in which 25 mol % of methyl acrylate is replaced by 25 mol % of methacrylic acid.

Table 4 summarizes the composition of polymers 9 to 14.

TABLE 4
Composition of Polymers 9 to 14

| | AA | AMA | EA | MA | Lipol 1 | DAP | DDM |
|---|---|---|---|---|---|---|---|
| Example 9 | 7.13 | 30 | 62 | | 0.87 | 0.2 | 0.1 |
| (Comparative) | | | | | | | |
| Example 10 | 15 | 30 | 52 | | 0.87 | 0.2 | 0.1 |
| (Invention) | | | | | | | |
| Example 11 | 27.76 | 11.58 | 59.82 | | 0.84 | 0.2 | 0.1 |
| (Comparative) | | | | | | | |
| Example 12 | 27.76 | 36.58 | 34.82 | | 0.84 | 0.2 | 0.1 |
| (Invention) | | | | | | | |
| Example 13 | 25.29 | 10.55 | | 63.4 | 0.76 | 0.2 | 0.1 |
| (Comparative) | | | | | | | |
| Example 14 | 25.29 | 35.55 | | 38.4 | 0.76 | 0.2 | 0.1 |
| (Invention) | | | | | | | |

The values of acrylic acid (AA), methacrylic acid (AMA), ethyl acrylate (EA), methyl acrylate (MA), and Lipol 1 are expressed in mol %. The values of diallyl phthalate (DAP) and n-dodecyl mercaptan (DDM) are expressed as mass %. The molecular weight of Lipol 1 is 1188 g/mol.

Lipol 1 is a lipophilically modified monomer having a linear saturated alkyl group of 18 carbon atoms connected via 20 residues of ethylene oxide to a methacryloyl group.

The polymers 9 to 14 are used in the hair gel composition of Table 2, and the gel compositions obtained are noted respectively as C′9 to C′14. The viscosity of the gels and their clarity were measured according to the same methods previously described. The results are reported in Table 5.

TABLE 5
Viscosity and Clarity of Styling Gels C′9 to C′14.

| Gel Composition | Viscosity CPS (20 tpm) | Clarity % T (420 nm) |
|---|---|---|
| C′9 (Comparative) | 67,000 | 84 |
| C′10 (Invention) | 70,000 | 87 |
| C′11 (Comparative) | 51,000 | 87 |
| C′12 (Invention) | 62,000 | 91 |
| C′13 (Comparative) | 56,000 | 89 |
| C′14 (Invention) | 63,000 | 93 |

The same resistance to humidity test as in part 3/is carried out, and the results of curl hold at 300 minutes are given in Table 6.

TABLE 6
Curl Hold of Compositions C′9 to C′14

| Gel Composition | Curl Hold in % |
|---|---|
| C′9 (Comparative) | 76 |
| C′10 (Invention) | 84 |
| C′11 (Comparative) | 65 |
| C′12 (Invention) | 85 |
| C′13 (Comparative) | 67 |
| C′14 (Invention) | 88 |

Increasing the proportion of acrylic acid from 7.13 mol % to 15 mol % in Example 9 results in more than a 10% improvement in curl hold. Increasing the proportion of acrylic acid from 11.58 mol % to 36.58 mol % in Example 11 results in more than a 30% improvement in curl hold. Increasing the proportion of acrylic acid from 10.55 mol % to 30.55 mol % in Example 13 results in more than a 31% improvement in curl hold.

These results make it possible to demonstrate the better performance of the polymers according to the invention, in particular, compared to those of the prior art.

1. An aqueous cosmetic composition for hair care comprising at least one setting resin, said resin comprising:
at least 30 mol % of monomer units of methacrylic acid and/or a salts thereof;
at least 10 mol % of monomer units of acrylic acid and/or maleic acid and/or itaconic acid, and/or a salt thereof;
the resin being water-soluble and having a weight-average molecular weight of 20,000 gm/mol or more.

2. The composition according to claim 1, wherein the resin comprises less than 80 mol % of the monomer units of methacrylic acid and/or a salt thereof.

3. The composition according to claim 1, wherein the resin comprises at least 20 mol % of the monomer units of acrylic acid and/or maleic acid and/or itaconic acid and/or a salt thereof.

4. The composition according to claim 1, wherein the resin comprises less than 70 mol % of the monomer units of acrylic acid and/or maleic acid and/or itaconic acid and/or a salt thereof.

5. The composition according to claim 1, wherein the resin is a copolymer of methacrylic acid and acrylic acid and/or their salts.

6. The composition according to claim 1, wherein the resin has a weight-average molecular weight greater than or equal to 60,000 g/mol.

7. The composition according to claim 1, wherein the resin has a weight-average molecular weight greater than or equal to 2,000,000 g/mol.

8. The composition according to claim 1, wherein the resin represents 0.05 to 10% by weight, based on the weight of the composition.

9. The composition according to claim 1, wherein the composition comprises:
water;
from 0.05 to 10% by weight of the setting resin; and
a thickening agent.

10. The composition according to claim 1, wherein the composition comprises a thickening agent consisting of a cross-linked polyacrylic acid.

11. The composition according to claim 1, wherein the composition is in gel form.

12. The composition according to claim 1, wherein the composition has a transmittance value greater than 85%, the transmittance measurement being carried out at 420 nm, with an optical path of 10 mm, at 25° C., for a solution comprising 2.4 g/L of resin in water.

13. A method for improving resistance to humidity of an aqueous cosmetic composition, said method comprising adding to an aqueous preparation a setting resin comprising:
at least 30 mol % of monomer units of methacrylic acid and/or a salt thereof;
at least 10 mol % of monomer units of acrylic acid and/or maleic acid and/or itaconic acid, and/or a salt thereof;
the resin being water-soluble and having a weight-average molecular weight of 20,000 g/mol or more.

14. A setting resin for an aqueous cosmetic composition for hair care, the resin comprising:
at least 30 mol % of monomer units of methacrylic acid and/or a salt thereof;
at least 10 mol % of monomer units of acrylic acid and/or maleic acid and/or itaconic acid, and/or a salt thereof;
the resin being water-soluble and having a weight-average molecular weight of 20,000 gm/mol or more.

15. The composition according to claim 1, wherein the resin comprises:
at least 30 mol % and less than 80 mol % of the monomer units of methacrylic acid and/or a salt thereof;
at least 20 mol % and less than 70 mol % of the monomer units of acrylic acid and/or maleic acid and/or itaconic acid and/or a salt thereof; and

16. The composition according to claim 15, wherein the resin is a copolymer of methacrylic acid and acrylic acid and/or their salts.

17. The composition according to claim 15, wherein the resin has a weight-average molecular weight greater than or equal to 60,000 g/mol.

18. The composition according to claim 16, wherein the resin represents 0.05 to 10% by weight, based on the weight of the composition.

19. The composition according to claim 16, wherein the composition is in gel form and comprises:
water;
a thickening agent; and
from 0.05 to 10% by weight of the setting resin, said setting resin having a weight-average molecular weight greater than or equal to 60,000 g/mol.

20. The composition according to claim 19, wherein the resin has a weight-average molecular weight greater than or equal to 2,000,000 g/mol.
